(19) United States
(12) Patent Application Publication
(10) Pub. No.: US 2004/0158153 A1
(43) Pub. Date: Aug. 12, 2004

(54) Ultrasonic probe for a stereoscopic image forming system

(75) Inventors: Felix Hirt, Wila (CH); Urs Moser, Zurich (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sulzer Markets and Technology AG, Winterthur (CH)

(21) Appl. No.: 10/757,090

(22) Filed: Jan. 13, 2004

(30) Foreign Application Priority Data
Feb. 10, 2003 (EP) 03405070.8

(51) Int. Cl. A61B 8/00

(52) U.S. Cl. 600/443; 600/459; 128/916

(57) ABSTRACT

The ultrasonic probe (1) is provided for a stereoscopic image forming system, which relates to a raster-like recording of data and its electronic processing. The probe includes groups (12) of sound transducers (11), which are arranged inside an elastomeric envelope (10) on a cylindrical carrier, rotatable around a longitudinal axis. The carrier is made up of at least two segments (3, 4). The segments are tiltable relative to one another through a tilting angle, and what is more about a tilting axis, which lies transversely to the longitudinal axis and preferably eccentrically to it. There is a stable positional state, in which the segments adopt a position with alignment in the direction of the longitudinal axis under the influence of a dynamic and/or static resetting force.

The invention relates to an ultrasonic probe for a stereoscopic image forming system, which can be used on a patient to produce three-dimensional reproductions of an organ from the interior of a body. The invention also relates to the use of this probe.

An ultrasonic measuring apparatus for an imaging process is known from EP-A-0 926 491. A probe of this apparatus is a rod-shaped body, which carries a plurality of transformer elements. The transformer elements are transducers, which transmit ultrasound signals and receive reflected components of these signals. A sound lobe is radiated from a transducer surface perpendicular to the surface. In a preferred embodiment of the probe the bar-shaped carrier is rotatable about a longitudinal axis.

The probe is developed for treatment procedures in which a catheter is introduced into the unopened beating heart of a patient and positioned there. The data required for a navigation of the catheter are gathered by means of the probe, which is introduced into the oesophagus. A stereo-scopic image has to be calculated from the data with a real time procedure, which has an adequate resolution, so that the operator is in a position at any time to know where the tip of the catheter is located. The transducers are arranged on the probe in groups. Switched electronically, only one group radiates the ultrasound signals at any one time and what is more the group which is respectively directed towards the heart from the continually rotating carrier. In order to maintain the required resolution in the image generation, three groups of eight transducers each are provided. There is the problem that a rigid probe, which is relatively long due to the large number of transducers, can not be swallowed by the patient.

The object of the invention is to produce an ultrasonic probe for a stereo-scopic image forming system, which can be introduced into the patient's oesophagus. This object is solved by the ultrasonic probe defined in claim 1.

The ultrasonic probe is provided for a stereoscopic image forming system, which relates to a raster-like recording of data and its electronic processing.

The probe includes groups of sound transducers, which are arranged inside an elastomeric envelope on a cylindrical carrier, rotatable around a longitudinal axis. The carrier is made up of at least two segments. The segments are tiltable relative to one another and what is more about a tilting axis, which lies transversely to the longitudinal axis and preferably eccentrically to it. There is a stable positional state in which the segments adopt a position with alignment in the direction of the longitudinal axis under the influence of a dynamic and/or static re-setting force.

The dependent claims 2 to 9 relate to advantageous embodiments of the probe in accordance with the invention. A use of the probe in accordance with the invention is the subject of claim 10.

The invention will be explained with the help of the drawings in the following. They show:

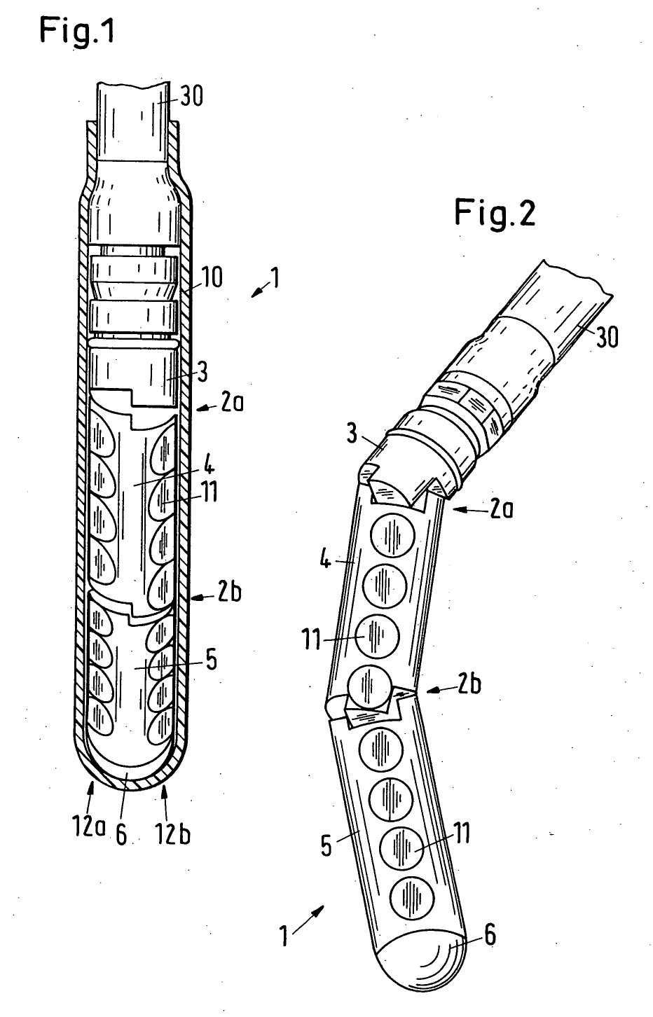

FIG. 1 a probe in accordance with the invention, the segments of which are tilted relative to one another—with a view perpendicular to the tilting axes,

FIG. 2 the probe in FIG. 1—without a protective envelope—from a different viewpoint,

FIG. 3 a development of the surface of the probe in one plane,

FIG. 4 a development for a second probe,

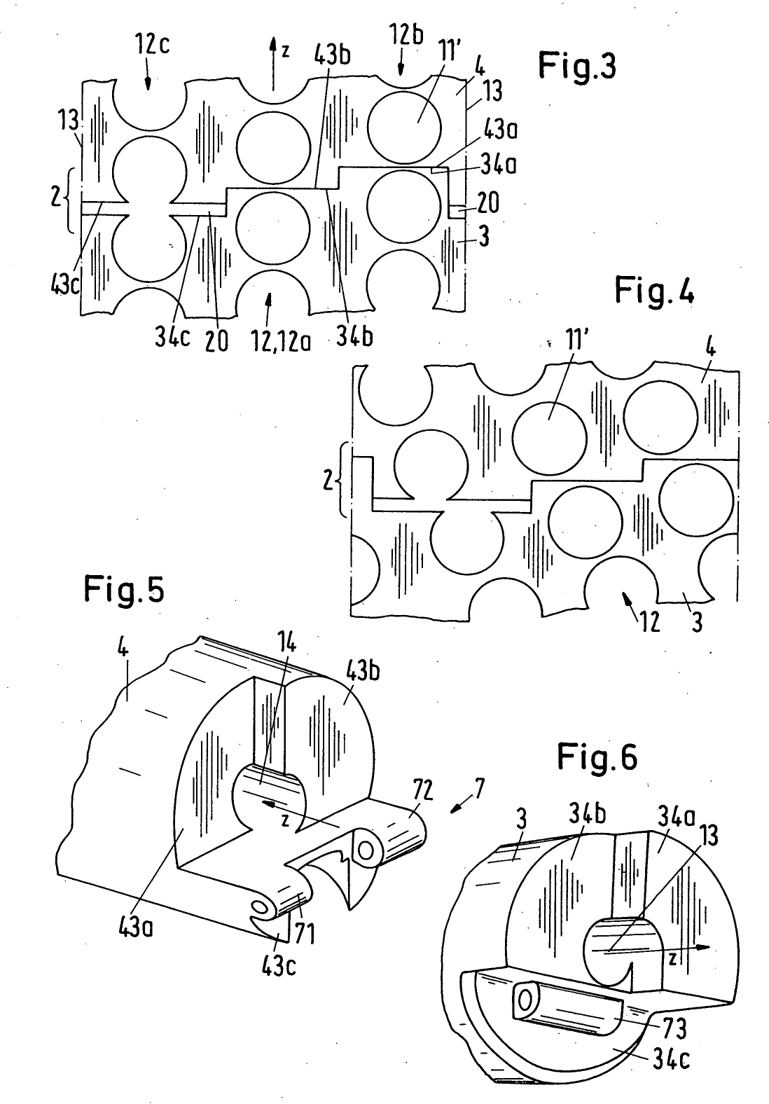

FIG. 5 a segment end with one half of a hinge,

FIG. 6 a segment end, which is the counterpiece of the segment end in FIG. 5,

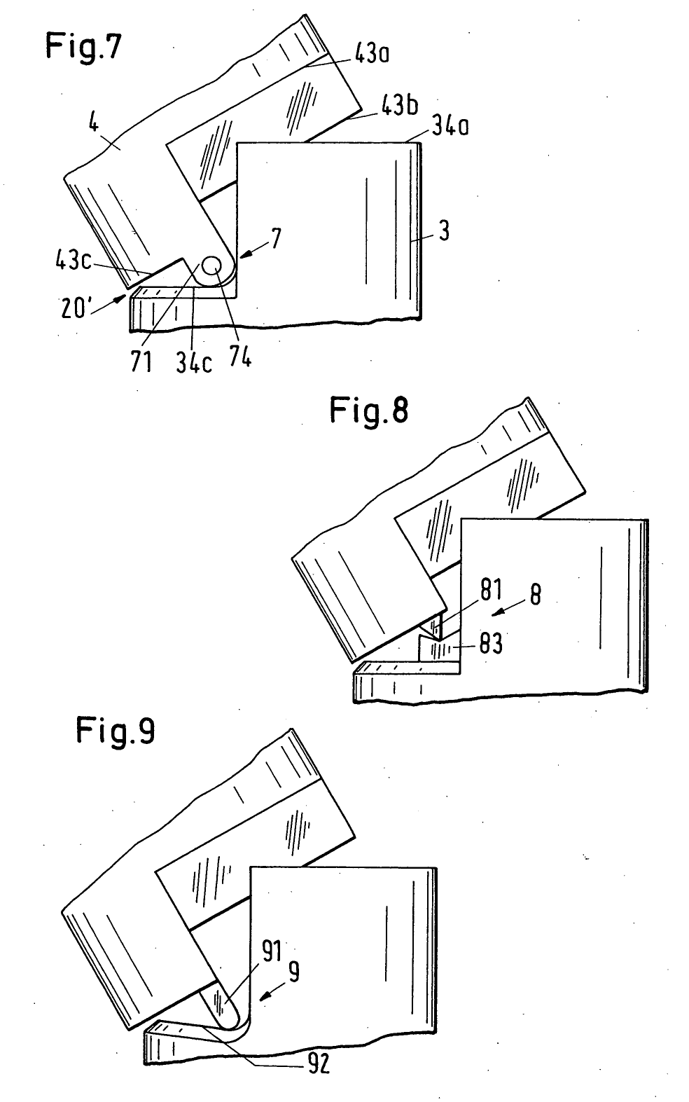

FIG. 7 a side view of the hinge of FIGS. 5 and 6,

FIG. 8 a first alternative to the hinge and

FIG. 9 a second alternative.

FIG. 1 represents an ultrasonic probe 1 in accordance with the invention for a stereoscopic image forming system which relates to a raster-like recording of data and its electronic processing. The probe 1, the diameter of which has to be smaller than 14 mm, includes sound transducers 11 (transducers), which are members of groups 12. The embodiment of the probe 1 which has been shown, the sound transducers 11 of which have a diameter of approximately 8 mm, includes three groups 12a, 12b and 12c, each having eight sound transducers 11; the group 12c is located on the side which can not be seen. The sound transducers 11 are arranged inside an elastomeric envelope 10 on a cylindrical carrier, rotatable around a longitudinal axis. The envelope 10 is a protective sleeve shown as a sectional view, which does not co-rotate with the carrier of the sound transducer 11. An oil forms a lubricating film between the carrier and the envelope 10. The carrier is made up of two segments 4 and 5. A further segment 3, which also co-rotates, contains a bearing necessary for the rotatability of the carrier. A second bearing is arranged in a tip 6, which does not co-rotate. The segments 3, 4, 5 are respectively tiltable relative to one another through a tilting angle by means of joints 2a and 2b.

Tilting axes of the joints 2a, 2b lie transversely to the longitudinal axis and in the illustrated example, eccentrically to this. The longitudinal axis is a z-axis (see FIGS. 3, 5 and 6), which extends from a probe connection 30 to the probe tip 6 when the segments 3, 4 and 5 are arranged in a straight line. In FIG. 1, in which the probe 1 in accordance with the invention is shown with a view perpendicular to the tilting axes, the segments 3, 4 and 5 are tilted relative to one another. There is a stable positional state in which the segments 3, 4 and 5 adopt a position with alignment in the direction of the longitudinal axis under the action of a resetting force.

In the example of FIG. 1 the elastomeric envelope 10 is able to exert a resetting force. It is however also possible that an elastic cable, which is inserted in a longitudinal channel of the segments, can produce the resetting force.

The probe 1 in accordance with the invention can include two or more segments on which sound transducers 11 are arranged. Each segment carries a number of sound transducers 11 distributed over the entire periphery, the number being equal to the number of groups 12 or a multiple of this.

FIG. 3 shows as a section a development of the probe surface, which in a stable positional state begins or ends at a longitudinal line 13 and which spreads out in one plane. The line 13 appears in the development as the two side lines drawn with chain-dotted lines, which are parallel to the z-axis. The positions of the sound transducers 11 are given by circles 11′. The groups 12, i.e. the three groups 12a, 12b and 12c extend in the z-direction, i.e. in the direction of the longitudinal axis. The groups 12 next to the sound transducers 11 are respectively offset in the z-direction by a distance which amounts to approximately a third of a transducer diameter. Thanks to this offset there is a better resolution of the image to be produced. However due to the offset a somewhat complicated shape has to be chosen for the joint 2 between the segments 3 and 4. The positional development of this shape is illustrated in FIGS. 5 and 6.

FIG. 5 shows one end of the segment 4 with one half of a hinge-like connection 7. FIG. 6 shows one end of the segment 3, which is the counterpiece to the segment 4 with the second half of the hinge 7. FIG. 7 shows the assembled hinge 7 in a side view. A connecting pin 74 journalled in two eyes 71 and 72 of the segment 4 forms the tilting axis. The connecting pin 74 is fixed in a holder 73 of the segment 3. Two regions of differing size are separated from each other between the neighbouring segments 3 and 4 by the eccentrically arranged tilting axis. After the hinge 7 has been put together, the two segments 3 and 4 form at least approximately a form-matched connection in the larger of these regions, when the segments 3 and 4 are in a stable positional state. In this form-matched connection part surfaces 43a and 43b come to lie on corresponding part surfaces 34a and 34b respectively. The contact points of these part surfaces can be recognised in the development of FIG. 3 as a stair-shaped line. A gap 20 remains between further part surfaces 43c and 34c. The maximum tilting angle is given by the width of this gap 20. This can amount to up to 30°. The position of the gap 20 is given in FIG. 7 with the reference numeral 20′.

In FIG. 4 there is a development for a second probe 1, in which the groups 12 are not aligned longitudinally, but rather the sound transducers 11 are arranged on helical curves.

FIG. 8 shows a first alternative to the hinge 7. The tilting axis is formed by a knife edge 81 which is supported in a notch 83. The knife edge 81 can be moved on the notch 83 in such a way that the necessary tilting positions can be produced. In order for the neighbouring segments 3 and 4 to remain connected with one another, an elastic cable has to be present (not illustrated). The cable is attached at a first end of the tip 6 and leads through the central channels 14, 13 for example (see FIGS. 5, 6) to the segment 3, where the second end of the cable is attached. The cable can also be inserted into an additional, not illustrated channel. The central channels 14, 13 are actually provided for electrical connection leads to the sound transducers 11.

A second alternative 9 is outlined in FIG. 9. Here an elastic cable is also required. The tilting axis is given through the line of contact between a flange 91, which has a round edge, and a flat support surface 92. On tilting, the round edge of the flange 91 rolls off on the support surface 92. At the same time the tilting axis clearly moves. The support surface 92 can include an angle relative to the longitudinal axis, which is smaller than 90°. In this case the tilting axis makes an additional movement in the direction of the longitudinal axis, whereby the resetting force of the cable is enhanced and thus the stability of the stable positional state of the segments 3 and 4 is increased.

A resetting force acting between the segments has to be relatively weak, so that the probe can be introduced into the oesophagus without much resistance. A resetting force of this kind is generally too weak to produce the stable positional state of the segments 3 and 4 in the measuring position. If the deviation from a straight alignment is not too much, then the carrier formed with the segments 3 and 4 can rotate. As soon as the carrier is rotated, an additional re-setting force forms dynamically, which forces the carrier into the straight line positional state. This positional state is thus completely stabilised by the rotation of the carrier (15 to 20 rotations per second). Thus a stable positional state is present, in which the segments adopt a position with alignment in the direction of the longitudinal axis under the action of a dynamic or static resetting force. The positional state can also be stabilised merely by means of a dynamic resetting force or by means of a static resetting force.

The sound transducer 11 has a diameter in the region of 6 to 10 mm. This diameter is preferably approximately 8 mm. The ratio between the diameters of the carrier and the sound transducer is in the range of 1.2 to 1.8; preferably this ratio amounts to approximately 1.5.

1. An ultrasonic probe (1) for a stereoscopic image forming system, which relates to a raster-like recording of data and its electronic processing, said probe including groups (12) of sound transducers (11), which are arranged inside an elastomeric envelope (10) on a cylindrical carrier, rotatable around a longitudinal axis, characterised in that the carrier is made up of at least two segments (3, 4), in that the segments are tiltable relative to one another through a tilting angle, and what is more about a tilting axis, which lies transversely to the longitudinal axis and preferably eccentrically to it, and in that there is a stable positional state in which the segments adopt a position with alignment in the direction of the longitudinal axis under the influence of a dynamic and/or static re-setting force.

2. A probe in accordance with claim 1, characterised in that the elastomeric envelope (10) exerts a re-setting force.

3. A probe in accordance with claim 1 or claim 2, characterised in that an elastic cable, which is inserted in a longitudinal channel (13, 14) of the segments (3, 4), exerts a re-settting force.

4. A probe in accordance with one of the claims 1 to 3, characterised in that the segments (3, 4) are rigid, a hinge-like connection (7) exists between two neighbouring segments and a connecting pin (74) journalled in two eyes of the hinge-like connection (71, 72) forms the tilting axis.

5. A probe in accordance with one of the claims 1 to 4, characterised in that two or more segments (3, 4, 5) are present and in that each segment carries a number of sound transducers (11), the number being equal to the number of groups (12) or a multiple of this, distributed over the whole periphery.

6. A probe in accordance with one of the claims 1 to 5, characterised in that more than two segments (3, 4, 5) which are tiltable relative to one other are present and that the tilting axes are aligned parallel to one another.

7. A probe in accordance with one of the claims 1 to 6, characterised in that the maximum tilting angle has a value of up to 30°.

8. A probe in accordance with one of the claims 1 to 7, characterised in that between neighbouring segments (3, 4) two regions of different size are separated from one another by the eccentrically arranged tilting axis and in that the two segments form at least approximately a form-matched connection in the larger of these regions, when they are in the stable positional state.

9. A probe in accordance with one of the claims 1 to 8, characterised in that three groups (12a, 12b, 12c) of sound transducers (11) are arranged on the carrier, in a fixed or variable direction respectively, which is parallel to the longitudinal axis or inclined towards this, in that each sound transducer has a diameter in the range of 6 to 10 mm, this diameter being preferably approximately 8 mm and in that the ratio between the diameters of the carrier and of the sound transducer is in the range of 1.2 to 1.8, preferably approximately 1.5.

10. The use of a probe in accordance with one of the claims 1 to 9, characterised in that on a human or animal body three-dimensional representations of a moving organ, in particular of the heart, are produced from an interior of the body, with an electronic image forming system and in real time, wherein the interior of the body is accessible from the outside, the interior of the body being the oesophagus in particular.
